(19) United States
(12) Patent Application Publication
(10) Pub. No.: US 2025/0114164 A1
(43) Pub. Date: Apr. 10, 2025

(54) ROTATING MARKER AND ADAPTER FOR IMAGE-GUIDED SURGERY

(71) Applicant: AUGMEDICS LTD., Yokneam Illit (IL)

(72) Inventors: Tomer Gera, Kfar Tavor (IL); Nissan Elimelech, Beerotaim (IL)

(21) Appl. No.: 18/930,558

(22) Filed: Oct. 29, 2024

Related U.S. Application Data
(62) Division of application No. 17/388,064, filed on Jul. 29, 2021, now Pat. No. 12,150,821.

(51) Int. Cl. A61B 34/20; A61B 17/00; A61B 17/70; A61B 90/00

(52) U.S. Cl. CPC A61B 90/39; A61B 2090/3916; A61B 2090/3966; A61B 2090/3983; A61B 2090/3991

(57) ABSTRACT

A patient marker couples to an anchoring device via a base having a base axis, base connections and a first indicator. The marker includes: an adapter having a first surface with connections configured to mate with the base connections, and a second surface with connections congruent with the base connections, and at least one second indicator. The marker includes an alignment target, having a target region with an alignment pattern, and a socket connected to the target region with socket connections congruent with the first surface connections. In a first configuration the socket couples to the base by mating the first surface connections with the base connections and mating the socket connections with the second surface connections. In a second configuration, the socket fits onto the base by mating the socket connections with the base connections. One of the indicators indicates a target orientation angle about the base axis.

### CROSS-REFERENCE TO RELATED APPLICATIONS

This application is a continuation of U.S. application Ser. No. 17/388,064, filed Jul. 29, 2021, which is hereby incorporated by reference herein in its entirety.

### FIELD OF THE INVENTION

The present invention relates generally to a marker and a marker adapter for image-guided surgery, and particularly to a marker that may be attached to a patient and to a marker adapter that allow multiple orientations of the marker.

### BACKGROUND OF THE INVENTION

During image guided surgery, it is important to register elements of a patient, upon whom the surgery is being performed, with equipment generating the image. This is typically the case where the surgery comprises an augmented reality system, which generates images of portions of the patient that are in registration with the actual portions.

In order to register the elements of the patient, and keep the registration, a patient marker may be fixedly attached to the patient. An initial image of a marker may be analyzed, and used to provide the registration. So long as a marker continues to be fixedly attached to the patient at the same location, the registration remains valid and may be used to track the patient. However, if the marker is moved and then re-attached, for example to provide access to the patient, the registration typically needs to be repeated.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides a patient marker for image guided surgery that is configured to be coupled to an anchoring device via a base, the base having a base axis, base connections and a first indicator, the patient marker including:
an adapter having a first surface having first surface adapter connections configured to mate with the base connections, and a second surface, opposite the first surface, having second surface adapter connections congruent with the base connections, and at least one second indicator; and
an alignment target, including:
a target region having an alignment pattern formed thereon; and
a socket connected to the target region and having socket connections congruent with the first surface adapter connections, so that:
in a first configuration of the marker, the socket is coupled to the base by mating the first surface adapter connections with the base connections and mating the socket connections with the second surface adapter connections, and
in a second configuration of the marker, the socket is fit onto the base by mating the socket connections with the base connections,
whereby an angle of orientation of the alignment target about the base axis is indicated by one of the first indicator and the at least one second indicator.

Typically, in any given configuration of the marker, only one of the first indicator and the at least one second indicator is accessible.

In a disclosed embodiment in the first configuration the alignment target fits to the adapter in a plurality of discrete orientations. The discrete orientations may consist of two orientations at 180° to each other.

In a further disclosed embodiment in the second configuration the alignment target fits to the base in a plurality of discrete orientations. The discrete orientations may consist of two orientations at 180° to each other.

In a yet further disclosed embodiment in the first configuration the angle of orientation includes a first pair of angles at 180° to each other, and in the second configuration the angle of orientation includes a second pair of angles at 180° to each other and at 90° to the first pair of angles.

In an alternative embodiment the base connections and the first surface adapter connections are configured so that the adapter mates with the base in one single orientation.

In a further alternative embodiment the at least one second indicator is a divot that acts as a verification point.

In a yet further alternative embodiment the at least one second indicator is formed as an extension of a surface of the adapter.

In a still further alternative embodiment one of the at least one second indicator is positioned so that it aligns with and prevents access to the first indicator, while the first surface adapter connections are mated with the base connections.

The at least one second indicator may consist of two indicators. The angle between lines from each of the two indicators to a central hole of the adapter may be obtuse.

The at least one second indicator may be configured to be touched by a tip of a tool used in the surgery.

There is further provided, according to an embodiment of the present invention, a method for producing a patient marker to be coupled to an anchoring device via a base, the base having a base axis, base connections and a first indicator, the method including:
configuring first surface adapter connections of an adapter having a first surface to mate with the base connections;
forming on a second surface of the adapter, opposite the first surface, second surface adapter connections congruent with the base connections, and at least one second indicator; and
providing an alignment target, consisting of:
a target region having an alignment pattern formed thereon, and
a socket connected to the target region and having socket connections congruent with the first surface connections, so that:
in a first configuration of the marker, the socket is coupled to the base by mating the first surface adapter connections with the base connections and mating the socket connections with the second surface adapter connections, and
in a second configuration of the marker, the socket is fit onto the base by mating the socket connections with the base connections,
whereby an angle of orientation of the alignment target about the base axis is indicated by one of the first indicator and the at least one second indicator.

There is further provided, according to an embodiment of the present invention, an adapter for providing additional orientations to a patient marker with respect to an anchoring device in image guided surgery, wherein the anchoring device has a base having a base axis and a first indicator, the adapter including:
a first surface configured to be coupled to the base;
a second surface, opposite the first surface, configured to be coupled to the patient marker; and
at least one second indicator,
wherein the patient marker is coupled to the base by coupling the first surface of the adapter with the base and coupling the patient marker with the second surface of the adapter, and wherein when the patient marker is coupled to the anchoring device via the adapter:
an angle of orientation of the patient marker about the base axis is indicated by only one of the at least one second indicator, and
the first indicator is not accessible.

Typically, the at least one second indicator consists of two indicators, and wherein when the patient marker is coupled to the anchoring device via the adapter, only one of the two indicators is accessible. Additionally or alternatively, the at least one second indicator may be configured to be touched by a tip of a tool used in the surgery and serve as a verification point.

The present invention will be more fully understood from the following detailed description of the embodiments thereof, taken together with the drawings in which:

### BRIEF DESCRIPTION OF THE DRAWING

FIG. 1 is a schematic illustration of a medical procedure, according to an embodiment of the present invention;

FIG. 2A illustrates elements of a patient marker, in an assembled and a partly exploded format, and FIGS. 2B, 2C, and 2D illustrate elements of portions of the marker, according to an embodiment of the present invention;

FIGS. 3A, 3B, 3C, and 3D illustrate four orientations of a target of the patient marker with respect to a clamp, according to an embodiment of the present invention;

FIG. 4 is a flowchart describing the use of the marker in the medical procedure, according to an embodiment of the present invention; and

FIG. 5 is a schematic illustration of a head-up display (HUD), according to an embodiment of the present invention.

### DETAILED DESCRIPTION OF EMBODIMENTS

### Overview

In an augmented reality system that is used for a medical procedure performed on a patient, the position of the patient in relation to an augmented reality assembly worn by a professional performing the procedure needs to be tracked, so that images presented in the assembly align with the patient. To implement the tracking, a patient marker, comprising optical elements, may be fixedly attached to the patient, and a camera in the assembly may be configured to image the elements. A processor may then analyze the image so as to track the marker. As long as the marker is fixed to the patient, the processor may use the tracking of the marker to track the patient.

However, during the procedure the patient marker may interfere with the professional's access to the patient, and/or the professional's view of the patient, necessitating adjustment of the patient marker.

Embodiments of the present invention provide a patient marker which may be adjusted, while not requiring any new image analysis of the optical elements of the marker to continue the tracking.

A disclosed embodiment of the present invention provides a patient marker comprising a base connecting to an anchoring device, herein by way of example assumed to comprise a clamp. The clamp has jaws configured to grip a bone of the patient, such as a spinous process. Once the jaws have gripped the bone of the patient, the base provides a rigid platform to which may be attached an alignment target that does not move relative to the patient's bone.

The alignment target may be attached, in a first configuration of the marker, directly to the base in one of a first plurality of preselected orientations. An image of the alignment target may then be acquired by the camera in the augmented reality assembly worn by the professional, to implement tracking of the target and of the patient. The target may be adjusted between the first plurality of preselected orientations, without requiring any new image analysis by the processor of the imaged target, so that the processor may continue the tracking.

To provide further orientations of the target, embodiments of the invention provide an adapter. Rather than attaching the target directly to the base, as in the first configuration described above, in a second configuration of the marker the target is attached to the adapter which is then attached to the base.

The adapter provides the target with a second plurality of preselected orientations, different from the first plurality. As for the first configuration, the target may be adjusted between the second plurality of preselected orientations, without requiring any new image analysis, so that the processor may continue the tracking.

In addition, the adapter is configured so that the first and second plurality of preselected orientations are simply related. In a disclosed embodiment the first plurality of configurations, which does not use the adapter, provides a first orientation wherein the target rotates 0° about a base axis and a second orientation wherein the target rotates 180° about the axis. The second plurality of configurations, which uses the adapter, provides a third orientation where the target rotates 90° about the axis and a fourth orientation where the target rotates 270° about the axis.

Since the alignment target may be attached, with or without use of the adapter, to the base in a multiplicity of preselected orientations, embodiments of the invention provide a simple and cost effective solution for adjusting the patient marker without requiring new image analysis of the target.

According to some aspects, an adapter, as described above, is provided, which allows further orientations to a marker. The adapter provides additional orientations to the patient marker with respect to the anchoring device. The base (referred to above) connecting to the anchoring device, or comprised in the anchoring device, is provided by the anchoring device, and the base has a base axis and a first indicator.

The adapter has two surfaces: a first surface that may be coupled to the base, e.g., by the first surface adapter connections, and a second surface, opposite the first surface, that may be coupled to the patient marker, e.g., via the second surface adapter connections. The adapter also has at least one, e.g., two, second indicators. The patient marker may be coupled to the base by coupling the first adapter surface to the base, and coupling the second adapter surface to the patient marker, so that the patient marker couples to the anchoring device via the adapter.

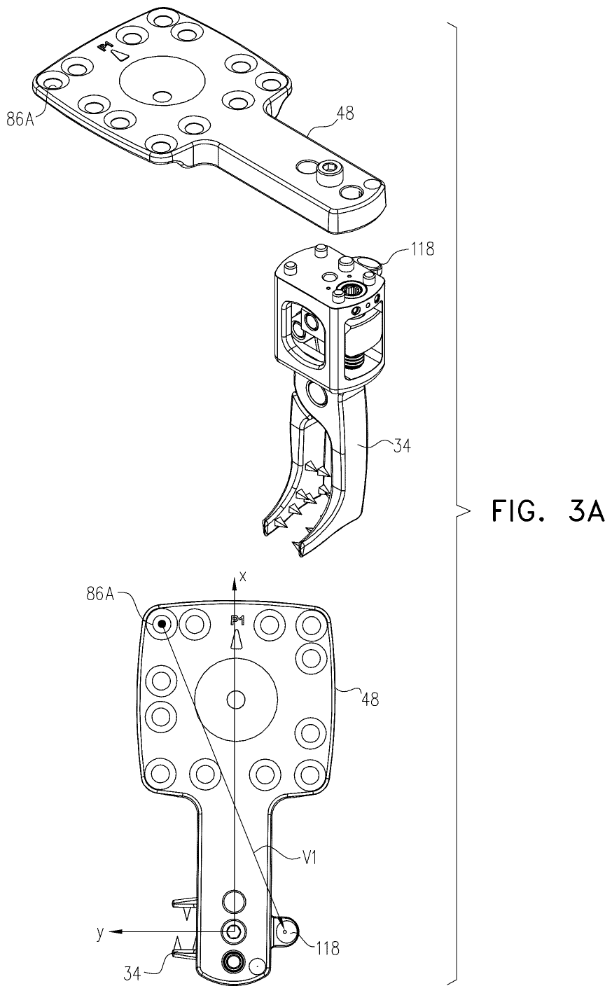

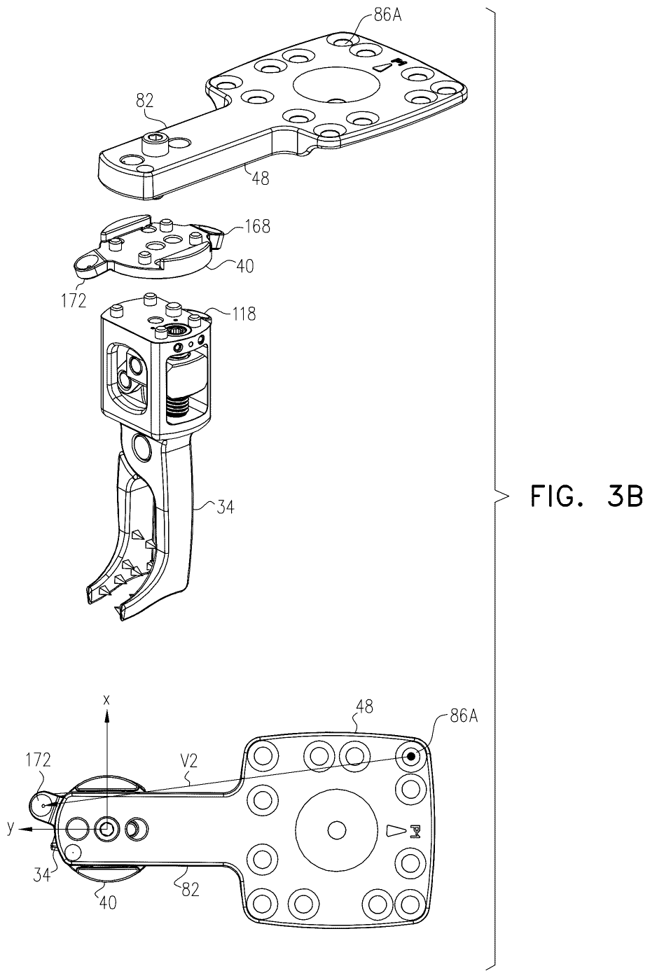

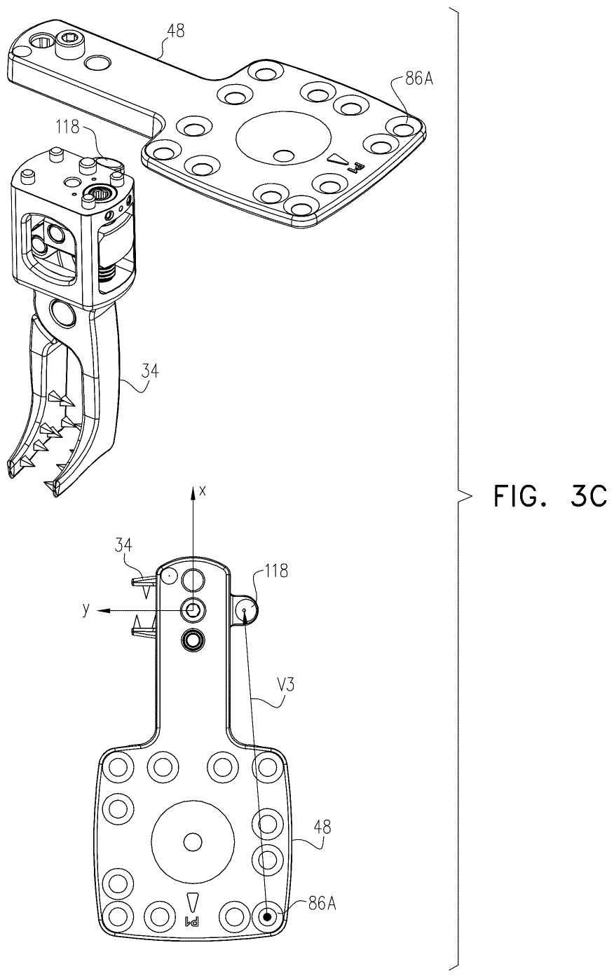

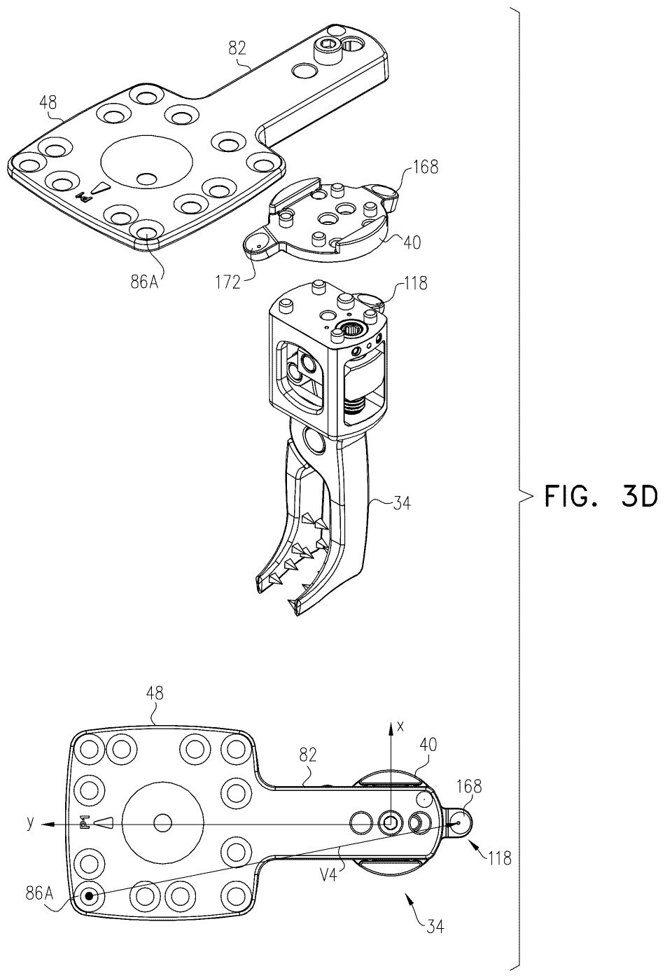

When the patient marker is coupled to the anchoring device as described above, the angle of orientation of the patient marker about the base axis is indicated by only one of the second indicators. For example, in case of two adapter indicators, when the marker is coupled to the adapter, only one indicator is accessible while the other is covered by the marker (e.g., positioned beneath the marker as exemplified in FIGS. 3B and 3D). Furthermore, the first indicator is not accessible (e.g., covered by the adapter, as shown in FIGS. 3B and 3D).

The adapter may be coupled to the anchoring device via its base and may be coupled to the marker; it may be formed, configured to and operate, all as detailed herein above and herein below, with the necessary changes.

During the medical procedure, in addition to tracking the patient marker, a position of the tip of a tool used in the procedure is also tracked. The base of the anchoring device has a verification point or divot fixedly located, in a known preselected position in the base. The adapter also has at least one, and herein by way of example two verification points, fixedly located in known preselected positions on the adapter.

To identify, during the procedure, which of the preselected orientations of the target is operative, the tool tip is placed on, or in proximity to, one of the verification points of the base or the adapter. The processor of the augmented reality system calculates a vector between the indicated verification point and the tracked alignment target, and from the vector (which is different for all the target orientations) identifies the orientation of the target.

### DETAILED DESCRIPTION

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

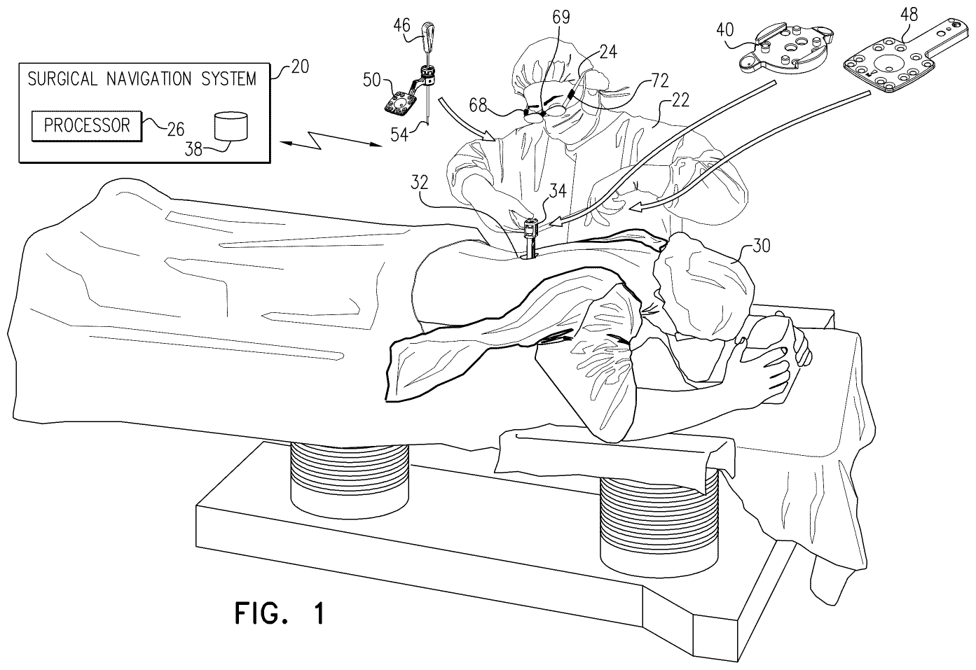

Reference is now made to FIG. 1, which is a schematic illustration of a medical procedure, according to an embodiment of the present invention. During the procedure, performed by a professional 22, the professional uses a surgical navigation system 20, which assists the professional in performance of the procedure. Surgical navigation system 20 comprises a processor 26, which operates elements of the system, and which communicates with an augmented reality assembly 24, comprising a set of augmented reality spectacles worn by professional 22, and that is incorporated in the system. Assembly 24 comprises, inter alia, an image capturing device 72, also termed herein a camera 72, that has a field of view 76 and that is configured to capture images in the visible spectrum. Assembly 24 also comprises an image capturing device 68, also termed a camera 68, that is operative in the non-visible spectrum, typically the infra-red, as well as a projector 69 that projects radiation in the operative spectrum of device 68. Functions of system 20, processor 26, and device 72 are described below. An assembly similar to augmented reality assembly 24, and its operation, are described in U.S. Pat. No. 9,928,629, to Benishti, et al., whose disclosure is incorporated herein by reference.

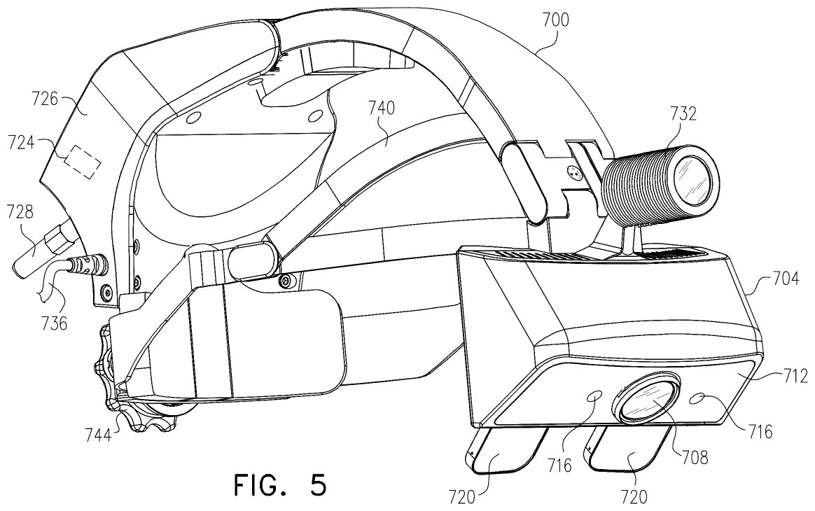

It will be understood that the present invention includes augmented reality assemblies other than assembly 24 described here. One example of such an alternative assembly is described with reference to FIG. 5, and all such assemblies are assumed to be comprised within the scope of the present invention.

In one embodiment processor 26 is assumed to be incorporated within a stand-alone computer, and the processor typically communicates with other elements of the system, including assembly 24, wirelessly, as is illustrated in FIG. 1. Alternatively or additionally, processor 26 may use optical and/or conducting cables for the communication. In further alternative embodiments processor 26 is integrated within assembly 24, or in the mounting of the assembly. Processor 26 is typically able to access a database 38, wherein are stored images and other visual elements used by system 20. Software enabling processor 26 to operate system 20 may be downloaded to the processor in electronic form, over a network, for example. Alternatively or additionally, the software may be provided on non-transitory tangible media, such as optical, magnetic, or electronic storage media.

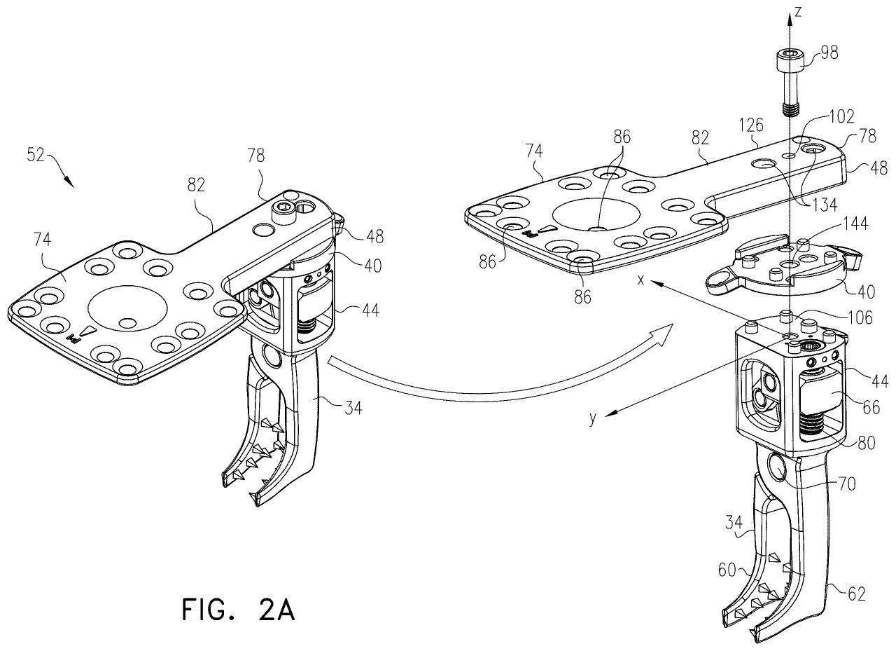

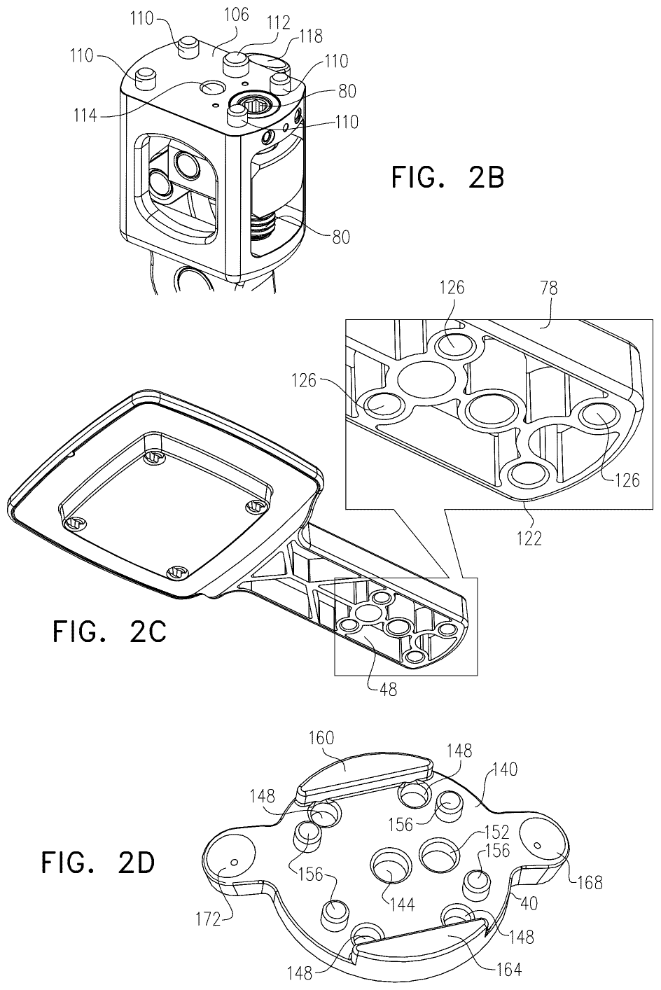

The medical procedure exemplified here is performed on a patient 30, and during an initial stage of the procedure professional 22 makes an incision 32 into the patient's back. The professional then inserts an anchoring device 34, herein assumed to comprise a spinous process clamp 34, into the incision, so that opposing jaws of the clamp are located on opposite sides of the spinous processes. The professional adjusts the clamp to grip one or more spinous processes, selected by the professional, of the patient. The professional may then attach an orientation adapter 40 to a base 44 (illustrated in FIG. 2A) of the clamp, and an alignment target 48 to the adapter, the target when attached to the base via the adapter operating as a patient marker 52 (illustrated in FIG. 2A). Patient marker 52 thus comprises alignment target 48 coupled, as described herein, to base 44. As is described below, processor 26 tracks alignment target 48 of patient marker 52, and the tracking is used by system 20 to determine the position and orientation of patient 30 during the medical procedure.

During the procedure professional 22 uses a tool 46 comprising a tool marker 50. Processor 26 also tracks tool 46 using tool marker 50 attached to the tool, and because the dimensions of the tool and the tool marker are known, the tracking provides the processor with the position of a tool tip 54 of the tool. A tool and tool marker similar to tool 46 and marker 50 are described in U.S. application Ser. No. 17/015,199, which is incorporated herein by reference.

During the procedure, it may be necessary for professional 22 to adjust the position of target 48, typically to enable the professional to more easily access elements of patient 30. Embodiments of the present invention facilitate such adjustment, by providing a plurality of different discrete orientations of alignment target 48 with respect to clamp 34. Each of the orientations may be identified by processor 26 from an acquired image of target 48 and from the tracked position of tool tip 54, as is described below. In addition, as is described below, once patient marker 52 has been through an initial setup process to align images presented in assembly 24 with patient 30, processor 26 may maintain the image alignment for the different identified orientation changes, by virtue of the fact that dimensions of target 48, adapter 40, and base 44 are known.

FIG. 2A illustrates elements of patient marker 52, in an assembled and a partly exploded format, and FIGS. 2B, 2C, and 2D illustrate elements of portions of the marker, according to an embodiment of the present invention. As stated above, marker 52 is formed by attaching alignment target 48 to adapter 40, which is in turn attached to base 44 of clamp 34. The clamp is described below.

Clamp 34 comprises a pair of jaws 60, 62 in a lower section of the clamp. The jaws are coupled to clamp base 44 in an upper section of the clamp, the base comprising a jaw adjustment mechanism 66. In the embodiment described herein, jaw 62 is fixed to base 44, and jaw 60 moves with respect to jaw 62, by being rotated about a hinge pin 70. Jaw adjustment mechanism 66 comprises an adjustment screw 80, which is coupled by a lever to jaw 60 so that rotation of the screw causes jaw 60 to approach or retreat from jaw 62. Thus professional 22 is able to cause the jaws of clamp 34 to grip or release a bone, such as one or more spinous processes, by rotating screw 80.

Alignment target 48 comprises a target region 74 and a socket 78, the target region and the socket being fixedly connected together by a connecting beam 82. Alignment target 48, together with its components target region 74 and socket 78, are generally planar. Target region 74, by way of example, is approximately rectangular and comprises optical elements 86. Elements 86 are arranged in a three-dimensional (3D) pattern, although in some embodiments the elements may be in a two-dimensional (2D) pattern, with no rotational axis of symmetry (other than a trivial axis of symmetry for rotating by 360°), and no mirror plane of symmetry, so that an image of the elements enables an unambiguous determination of the location and orientation of the target region. Elements 86 are typically retroreflectors. An entity with an arrangement of optical elements similar to the arrangement herein is described in U.S. patent application Ser. No. 16/901,026, which is incorporated herein by reference.

For clarity, in the description herein, elements of marker 52 are assumed, by way of example, to be referenced to a Cartesian set of orthogonal xyz axes, with origin at the center of an upper plane surface 106 of base 44, as illustrated in FIG. 2A. A z-axis is assumed to be orthogonal to plane surface 106, a y-axis is assumed to be parallel to a symmetry axis of hinge 70, and an x-axis is assumed to be orthogonal to the y and z axes.

As stated above, socket 78 is generally planar, and has a central hole 102. As is explained below, socket 78 and adapter 40 are fixed to base 44 by a captive screw 98 that penetrates a socket central hole 102 and also an adapter central hole 144. Holes 102 and 144 are dimensioned as clearance holes for screw 98.

FIG. 2B illustrates upper surface 106 of base 44. Surface 106 comprises a threaded hole 114 at the center of the surface, that accepts screw 98. Surface 106 also comprises four substantially similar cylindrical protuberances 110 located at the corners of a non-square rectangle, and distributed symmetrically about hole 114. As described below, protuberances 110 act as connections for base 44. There is a cylindrical protuberance 112 that in the illustrated embodiment is centered on a longer side of the non-square rectangle, and that is larger than protuberances 110. Extending from base 44, and fixed to the base, is a divot 118 that acts as a verification point or indicator and that is herein also termed divot indicator 118 or just indicator 118. There is an aperture in surface 106 that provides access to screw 80.

FIG. 2C illustrates a lower surface 122 of socket 78, and an upper surface of the socket is shown in FIG. 2A. Lower surface 122 comprises four cylindrical recesses 126 that are dimensioned and positioned to accept protuberances 110 of surface 106, when socket 78 is placed on surface 106, so that recesses 126 also act as connections. An upper surface 126 of the socket comprises two access holes 134, distributed symmetrically about clearance hole 102. In addition, when socket 78 is mated with surface 106, clearance hole 102 aligns with threaded hole 114, so that screw 98 may be used to attach the socket, and thus the target, to base 44.

The description above, of target 48 being directly attached to surface 106, i.e., without adapter 40 being used, corresponds to a first configuration of marker 52. It will be understood that because of the rectangular symmetry of recesses 126 and protuberances 110, there are two possible orientations of target 48 with respect to clamp 34, when the target is attached to base 44 in this first configuration. A partially exploded view of each orientation is shown in FIG. 3A and FIG. 3C.

FIG. 2D illustrates an upper surface 140 of adapter 40. Adapter 40 is generally circular, and clearance hole 144 is located at the center of the adapter. Adapter 40 comprises four cylindrical holes 148 that are dimensioned and positioned to accept protuberances 110 of surface 106, when the adapter is placed on surface 106. Holes 148 are distributed symmetrically about central clearance hole 144, and it will be understood that holes 148 form a non-square rectangle. Holes 148 penetrate to a lower surface of adapter 40, and act as connections. To enable the placement referred to above, adapter also comprises a fifth cylindrical hole 152, that is dimensioned and positioned to accept protuberance 112 when holes 148 connect with, i.e., mate with, protuberances 110.

Four cylindrical protuberances 156 are formed on surface 140, and they are arranged to be congruent to protuberances 110, and thus lie in a non-square rectangle. Protuberances 156 are distributed symmetrically about central hole 144, and are located so that the non-square rectangle they form is orthogonal to the non-square rectangle formed by holes 148. As for protuberances 110, protuberances 156 act as connections.

In operation of marker 52 (illustrated in FIGS. 2A-2D) using adapter 40, the adapter is first placed on surface 106 of base 44 so that holes 148 mate with protuberances 110, and so that hole 152 mates with protuberance 112. It will be understood that for this mating there is only one possible orientation of the adapter with respect to base 44, and that in this orientation clearance hole 144 aligns with threaded hole 114 of the base.

Once adapter 40 has been placed on surface 106, socket 78 of target 48 may be mated with the upper surface of the adapter, by pushing recesses 126 of the socket onto protuberances 156. The mating is facilitated by forming retaining shoulders 160, 164 on the surface of the adapter, symmetrically on either side of hole 144, the shoulders having internal walls that are separated by the width of socket 78, together with a clearance value.

The description above, of target 48 being attached to surface 106 via adapter 40, corresponds to a second configuration of marker 52. It will be understood that in this second configuration, there are two possible orientations of target 48 with respect to clamp 34, when the target is attached to adapter 40. A partially exploded view of each orientation of the second configuration is shown in FIG. 3B and FIG. 3D.

First and second divots 168, 172, which act as verification points or indicators and which are also herein termed first and second divot indicators 168, 172, or just indicators 168, 172, are formed in adapter 40 as extensions of surface 140. First indicator 168 is positioned so that it aligns with, and prevents access to, indicator 118 when the adapter is attached to surface 106. Second indicator 172 is positioned so that the angle between a line segment from indicator 172 to central hole 144 and a line segment from indicator 168 to the central hole is an obtuse angle, less than 180°. In a disclosed embodiment the angle is approximately 160°, but other angles are possible. Positioning of second indicator 172 is referred to further below.

As stated above, marker 52 has two possible configurations, and for each configuration target 48 has two possible orientations with respect to clamp 34. There are thus four different orientations of target 48 with respect to the clamp. As is described below, using images of optical elements 86 of target 48, and being provided with the positions of indicators 118, 168, or 172, processor 26 is able to identify in which of the four orientations target 48 is. As is also described below, the processor is able to adjust the images presented to the professional in the augmented reality assembly to compensate for any change in the orientation.

FIGS. 3A, 3B, 3C, and 3D illustrate the four possible orientations of target 48 with respect to clamp 34, according to an embodiment of the present invention. In each of the figures, marker 52 is drawn as a partially exploded view of the marker, and also as a top-down, i.e., down the z-axis, view of the marker. From consideration of the figures, and of the construction of the elements of marker 52 described above, it will be understood that in each of the four orientations target 48 is rotated clockwise by a multiple of 90° from the x-axis, i.e., in FIG. 3A by 0°, in FIG. 3B by 90°, in FIG. 3C by 180°, and in FIG. 3D by 270°. The orientations are also referred to herein as the 0°, 90°, 180°, and 270° orientation.

In the first configuration of the marker, i.e., when adapter 40 is not used, corresponding to FIGS. 3A and 3C and the 0° and 180° orientations, the only indicator present, and thus accessible, is indicator 118 of base 44.

In the second configuration of the marker, when adapter 40 is used, corresponding to FIGS. 3B and 3D and the 90° and 270° orientations, all three indicators 118, 168, and 172, are present. However, the indicators of adapter 40 are arranged so that only one of indicators 118, 168, and 172 is accessible. Thus, in the 90° orientation illustrated in FIG. 3B, indicators 118 and 168 are obscured by connecting beam 82, and only indicator 172 is accessible. In the 270° orientation illustrated in FIG. 3D, indicator 172 is obscured by connecting beam 82, and indicator 168 obscures indicator 118 of base 44, so that only indicator 168 is accessible.

It will be appreciated that in each of the four possible orientations, only one indicator is accessible.

Using the locations of the accessible indicators, for each orientation processor 26 calculates a respective vector from a preselected point on target 48 to the accessible indicator. In the figures, the preselected point is assumed to be an optical element 86 at a corner of target 48, herein termed element 86A, but any other convenient preselected point on the target, such as a center of the target, may be used. As shown in the figures, for orientations 0°, 90°, 180°, and 270° there are respective vectors V1, V2, V3, and V4.

Table I below illustrates the orientations, vectors and parameters associated with the vectors. The vector parameters are assumed to comprise ordered triples, as measured with respect to the xyz axes described above.

TABLE I

| Orientation | Vector | Vector Parameters |
|---|---|---|
| 0° | V1 | (x₁, y₁, z₁) |
| 90° | V2 | (x₂, y₂, z₂) |
| 180° | V3 | (x₃, y₃, z₃) |
| 270° | V4 | (x₄, y₄, z₄) |

Table II below gives a numerical example, according to a disclosed embodiment, of approximate vector parameters, in mm., for the different orientations.

TABLE II

| Orientation | Vector | Vector Parameters |
|---|---|---|
| 0° | V1 | (−103, −42, −13) |
| 90° | V2 | (−17, +124, −8) |
| 180° | V3 | (+103, +8, −13) |
| 270° | V4 | (+25, −126, −8) |

Since the dimensions of all elements of marker 52 are known, numerical values of the vectors of the different possible orientations may be precalculated and stored by processor 26. The vectors may be stored in any convenient format known in the art, for example related to a cylindrical coordinate system, or to a spherical coordinate system, or to another Cartesian set of axes, rather than the Cartesian axes presented here. All such formats are assumed to be comprised within the scope of the present invention.

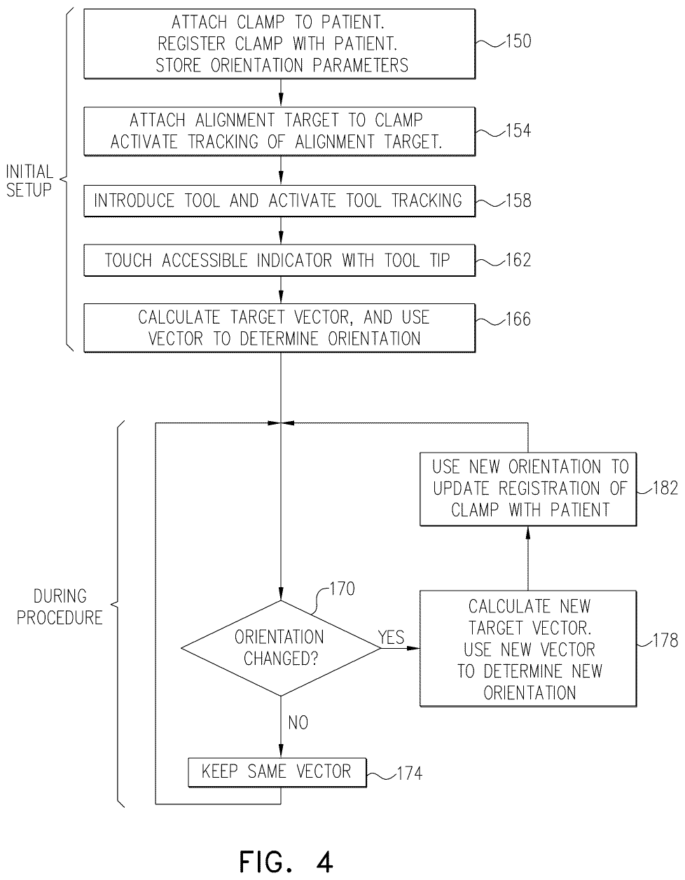

FIG. 4 is a flowchart describing the use of marker 52 in the medical procedure referred to above, according to an embodiment of the present invention.

In an initial step 150, professional 22 attaches clamp 34 to a bone of patient 30, herein assumed to comprise a spinous process of the patient, by rotating screw 80. The clamp is registered to the patient, typically by a CT (computerized tomography) scan. A method for registering a clamp to a patient is provided in U.S. patent application Ser. No. 16/524,258, which is incorporated herein by reference. Also in the initial step, the preselected point on the target (referred to above) and vector parameters for the four orientations of marker 52, corresponding to the values in Table I, are stored by processor 26.

In a target attachment step 154, the professional uses screw 98 to attach alignment target 48 to the clamp, in one of the four orientations illustrated in FIGS. 3A-3D. It will be understood that depending on the orientation selected, the professional may or may not use adapter 40 for the attachment.

Once alignment target 48 is attached to the clamp, camera 68 and projector 69 are activated, so that processor begins to track the alignment target and the preselected point in the target.

In a tool activation step 158, professional 22 introduces tool 46 into proximity with the patient, and initiates tracking of the tool. The tracking uses tool marker 50, and images of the marker generated in camera 68 from radiation of projector 69. The tracking provides processor 26 with the position of tool tip 54.

In an indicator access step 162, professional 22 touches tool tip 54 on the single indicator, i.e., indicator 118, 168, or 172, that is accessible by virtue of the orientation of target 48 formed in step 154. Processor 26 may recognize that the indicator is being touched by any convenient method known in the art, for example by the tool tip being held on, or in proximity to, the indicator for a preselected period of time, and/or by the tool tip being within a preselected region of the tracked alignment target.

In an orientation calculation step 166, from coordinates of the accessible indicator position and of the preselected point position in the tracked alignment target the processor calculates coordinates of the vector parameters (as in Table I) joining the positions, and from the parameters stored in step 150, identifies the orientation of step 154.

Step 166 completes an initial setup set of steps of the flowchart. The remaining steps are performed as the procedure being performed by professional 26 continues.

Control in the flowchart passes to a decision step 170, where the processor iteratively checks if a change of orientation of the alignment target has occurred. Typically, the change is effected by professional 26 removing then re-attaching the target to clamp 34.

The processor may perform its check by issuing a request, after a preset time period has passed, to the professional to touch tool tip 54 on the single accessible indicator. Alternatively or additionally the professional may inform the processor that a change has been made by any convenient method known in the art, for example by placing tool tip 54 on the single accessible indicator for the preselected period of time referred to above.

If decision step 170 returns negative, i.e., the processor finds there is no change in the target orientation, then in a no change step 174, the processor continues using the existing vector, and control returns to decision step 170.

If decision step 170 returns positive, i.e., the processor finds there is a change in the target orientation then in a change vector step 178 the processor calculates the new coordinates of the new target vector, as described in step 166. The processor uses the new vector to determine the new orientation of the target, and uses the new orientation to continue tracking the target.

In an update registration step 182, the processor uses the new orientation to update the registration of clamp 34 with patient 30. Control then returns to decision step 170.

FIG. 5 is a schematic figure illustrating a head-up display (HUD) 700, according to an embodiment of the present invention. HUD 700 is worn by professional 22, and may be used in place of assembly 24 (FIG. 1). HUD 700 comprises an optics housing 704 which incorporates an infrared camera 708. Housing 704 also comprises an infrared transparent window 712, and within the housing, i.e., behind the window, are mounted one or more infrared projectors 716. Mounted on housing 704 are a pair of augmented reality displays 720, which allow professional 22 to view entities, such as part or all of patient 30 through the displays, and which are also configured to present to the professional images that may be received from database 38 or any other information.

The HUD includes a processor 724, mounted in a processor housing 726, which operates elements of the HUD. Processor 724 typically communicates with processor 26 via an antenna 728, although in some embodiments processor 724 may perform some of the functions performed by processor 26, and in other embodiments may completely replace processor 26.

Mounted on the front of HUD 700 is a flashlight 732. The flashlight projects visible spectrum light onto objects so that professional 22 is able to clearly see the objects through displays 720. Elements of the head-up display are typically powered by a battery (not shown in the figure) which supplies power to the elements via a battery cable input 736.

HUD 700 is held in place on the head of professional 22 by a head strap 740, and the professional may adjust the head strap by an adjustment knob 744. It will be understood that although the above description refers to a group of discrete orientations comprising the angles of 0°, 90°, 180°, and 270°, those having ordinary skill in the art will be able to adapt the description, mutatis mutandis, for other groups of discrete orientations, such as 0°, 60°, 120°, and 180°, and all such groups are considered to be comprised within the scope of the present invention.

While the description above assumes that anchoring device 34 comprises a clamp, it will be understood that the anchoring device may comprise other types of attachment to the bone of patient 30, such as a pin that is inserted into the bone. Thus, the scope of the present invention includes all such types of attachment.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1-20. (canceled)

21. An adapter for providing additional orientations to a patient marker with respect to an anchoring device in image guided surgery, wherein the anchoring device comprises a base having a base axis and a first indicator, the adapter comprising:
a first surface configured to be coupled to the base;
a second surface, opposite the first surface, configured to be coupled to the patient marker; and
at least one second indicator,
wherein the patient marker is coupled to the base by coupling the first surface of the adapter with the base and coupling the patient marker with the second surface of the adapter, and wherein when the patient marker is coupled to the anchoring device via the adapter:
an angle of orientation of the patient marker about the base axis is indicated by only one of the at least one second indicator, and
the first indicator is not accessible.

22. The adapter of claim 21, wherein the at least one second indicator comprises two indicators, and wherein when the patient marker is coupled to the anchoring device via the adapter, only one of the two indicators is accessible.

23. The adapter of claim 21, wherein the at least one second indicator is configured to be touched by a tip of a tool used in the surgery and serve as a verification point.

24. The adapter of claim 23, wherein the at least one second indicator comprises a divot that serves as the verification point.

25. The adapter of claim 24, wherein the divot is formed in an extension of the second surface.

26. The adapter of claim 22, wherein an angle between lines from each of the two indicators to a central hole of the adapter is obtuse.

27. The adapter of claim 22, wherein each of the two indicators of the at least one second indicator comprises a divot that serves as a verification point configured to be touched by a tip of a tool used in the surgery.

28. The adapter of claim 21, wherein the first surface comprises first surface adapter connections configured to mate with the base, and the second surface comprises second surface adapter connections configured to mate with the patient marker.

29. The adapter of claim 28, wherein at least one of the first surface adapter connections or the second surface adapter connections comprises holes configured to mate with protuberances.

30. The adapter of claim 28, wherein one of the first surface adapter connections or the second surface adapter connections comprises holes configured to mate with protuberances, and the other of the first surface adapter connections or the second surface adapter connections comprises protuberances configured to mate with holes.

31. The adapter of claim 28, wherein the second surface adapter connections are distributed symmetrically about a central hole of the adapter.

32. The adapter of claim 31, wherein the second surface adapter connections are arranged in a non-square rectangle.

33. An adapter for providing additional orientations to a patient marker with respect to an anchoring device in image guided surgery, wherein the anchoring device comprises a base having a base axis and a first indicator, the adapter comprising:
a first surface comprising first surface adapter connections configured to mate with the base;
a second surface, opposite the first surface, comprising second surface adapter connections, the second surface adapter connections distributed symmetrically about a central hole of the adapter such that the second surface adapter connections are configured to mate with the patient marker in at least a first orientation and a second orientation; and
at least two second indicators,
wherein the patient marker is coupled to the base by coupling the first surface of the adapter with the base and coupling the patient marker with the second surface of the adapter, and wherein when the patient marker is coupled to the anchoring device via the adapter:
only one of the at least two second indicators is accessible, with a first of the at least two second indicators being accessible in the first orientation and a second of the at least two second indicators being accessible in the second orientation.

34. The adapter of claim 33, wherein when the patient marker is coupled to the anchoring device via the adapter the first indicator is not accessible.

35. The adapter of claim 33, wherein each of the at least two second indicators comprises a divot configured to serve as a verification point configured to be touched by a tip of a tool used in the surgery.

36. The adapter of claim 35, wherein the divot is formed in an extension of the second surface.

37. The adapter of claim 33, wherein an angle between lines from each of two of the at least two second indicators to the central hole of the adapter is obtuse.

38. The adapter of claim 33, wherein at least one of the first surface adapter connections or the second surface adapter connections comprises holes configured to mate with protuberances.

39. The adapter of claim 33, wherein one of the first surface adapter connections or the second surface adapter connections comprises holes configured to mate with protuberances, and the other of the first surface adapter connections or the second surface adapter connections comprises protuberances configured to mate with holes.

40. The adapter of claim 33, wherein the second surface adapter connections are arranged in a non-square rectangle.
